(19) United States
(12) Patent Application Publication
(10) Pub. No.: US 2009/0246243 A1
(43) Pub. Date: Oct. 1, 2009

(54) Carbonaceous Protective Multifunctional Coatings

(75) Inventors: Ludvik Martinu, Montreal (CA); Jolanta Ewa Klemberg-Sapieha, Pointe Claire (CA)

Correspondence Address: MEDTRONIC, INC., 710 MEDTRONIC PARKWAY NE, MINNEAPOLIS, MN, 55432-9924, US

(73) Assignee: La Corporation de I'Ecole Polytechnique, Montreal (CA)

(21) Appl. No.: 12/054,623

(22) Filed: Mar. 25, 2008

(51) Int. Cl. C23C 28/00; A61F 2/00

(52) U.S. Cl. 424/423; 427/2.24; 427/2.1

(57) ABSTRACT

The present invention relates to Diamond-like Carbon (DLC) coatings on metal substrates and methods of making the same. The invention particularly relates, in a preferred embodiment, to a metallic substrate such as stainless steel or Ti alloy having a silicon material (such as an organosilicone, silicon nitride, silicon carbide or silicon carbon nitride) intermediate layer and a DLC layer, optionally doped with Si, deposited thereon, and a method for making the same.

### FIELD OF INVENTION

The present invention generally relates to carbonaceous coatings on metal substrates and methods of making the same, and specifically, to Diamond-like Carbon (DLC) coatings for medical devices having good adhesion and methods of making the same.

The invention particularly relates, in a preferred embodiment, to a metallic substrate such as stainless steel or Ti alloy having a silicon material (such as an organosilicone, silicon nitride, silicon carbide or silicon carbon nitride) intermediate layer and a DLC layer, optionally doped with Si, deposited thereon, and a method for making the same.

### BACKGROUND

The unique physical, chemical, electrical, mechanical, tribological, optical, biocompatibility and other properties of amorphous diamond like carbon (DLC), and doped or nano-structured DLC, together with the potential to adjust the properties by choosing appropriate deposition parameters, make DLCs suitable for a variety of applications. Exemplary applications include coatings on cutting tools, such as shaving blades and surgical knives, wear resistant components, body implants, computer memory discs, infrared windows, optical devices, sunglasses and ophthalmic lenses, micro-electro-mechanical systems (MEMS), sensors, flat panel displays, cold cathodes, sports equipment and specific functional or protective coatings with tailored surface characteristics.

DLC films were first deposited by quenching a beam of C⁺ ions accelerated in a vacuum of 10⁻⁶ Torr to a negatively biased substrate. The attractive properties of the materials have stimulated a large amount of research on their deposition and characterization, and on the development of relevant applications. The properties of the films are affected by preparation conditions, and, for a given preparation system, depend on the amount of hydrogen incorporated in the films. Some of that hydrogen can be incorporated as unbound hydrogen, and the relative fraction of bound or unbound hydrogen also affects the properties of the films. Although the type of hydrocarbon used as a precursor often appears to have only an insignificant effect on film properties, this is not always true. The properties of DLC can be tailored by adjusting deposition parameters such as energy of the bombarding ions, substrate bias voltage and temperature, power density, and precursor. In addition, the incorporation of atoms, molecules and nanometer size particles can substantially enhance the performance of such carbon-based films and open avenues leading to new applications, devices and value-added products.

The wear resistance of DLC films also appears to be strongly dependent upon their deposition conditions. It can be enhanced while modifying the material through incorporation of different elements. In spite of the high chemical inertness of DLC films, their tribological behavior is controlled to a large extent by their surface chemistry. The tribo-chemical behavior of DLC films (i.e., the effect of environment on their friction and wear) is quite well known. One major drawback of hard DLC films is the excess build-up of internal stress at thickness levels exceeding a few hundred nanometers. Several methods have been attempted to reduce internal stresses in DLC films. These methods include: (i) the incorporation of dopants to the film network, (ii) supplying the necessary ion bombardment energy in terms of high ion fluxes, and (iii) alternating high and low stress sublayers.

Doping can also lead to an increased stability at elevated temperatures and to modification of the surface energy of the material. Addition of N has been found to give rise to higher electrical conductivity, while incorporation of metals can give rise to interesting electrical and optical (color) effects and magnetic properties. Elements such as Cu, Ag, and V, embedded in the DLC matrix, were found useful for the preparation of surfaces with a tunable antibacterial effect. Addition of F, Al or Fe has been found to decrease surface energy, while doping with Ti or Cu has been found to reduce compressive stress and improve wear resistance. It has been shown that incorporation of F, O, N, and Si in DLC could modify water wetting angles of the DLC films. The incorporation of F or Si has been found to increase the contact angle of water, while the incorporation of O and N has been found to decrease the contact angle. The effect of F and Si on the surface energy of DLC films was believed to be attributed to the reduction primarily of the polar part of the surface energy, due to the loss of sp² C hybridization and dangling bonds. Yet DLC films containing F and Si can be prepared to be as wear resistant as the pure hydrogenated DLC. The incorporation of Si in DLC films has been reported to render their friction against steel and the wear of the steel counterpart insensitive to moisture. DLC films coated on metal substrates and containing 15% Ta, W, Ti, or Nb, having wear resistance similar to that of DLC and friction coefficients <0.2, but higher conductivity (up to 0.005 Ω/cm), have been deposited by dc magnetron sputtering of the metals in acetylene.

The largest use of DLC coatings for medical application is in orthopedics and articulated prostheses, orthopedic pins, joint replacements and an intraocular lens. DLC coatings have been proposed for artificial heart valves, catheters, stents, as well as root implants for teeth, dental instruments, and surgical scalpels.

Another important factor that influences the protection characteristics of DLC films is their adhesion to the substrate. It has been determined that a bond layer contributes to the adhesion of the DLC films to the substrates. For example, a simple nitrided bond layer has been reported to provide better adhesion properties and dry wear resistance of DLC on SS316L, and an amorphous hydrogenated silicon (a-Si:H) bond layer on Ti-6Al-4V has also been reported. However, it was reported that there are always pores, even in good quality DLC films. Even if DLC can be tuned to provide individual functional properties including wear, corrosion resistance, electrical conductivity, adhesion, surface energy, biocompatibility etc., the problem to provide all of them simultaneously when using metal substrates has not been solved.

Thus, the use of DLC has been often limited by their incompatibility with the substrate materials and by their thermal and environmental stability. In fact, one major problem with the application of DLC on implantable devices is the poor adhesion to metals and polymers in the body fluids which may lead to corrosion and formation of harmful debris.

Thus what is needed is a DLC coating for a medical application that overcomes these limitations.

### SUMMARY OF INVENTION

The present invention is directed to a DLC system to enhance a device's tribological performance in a biological environment (e.g. body-fluid) with an intermediate layer (sandwiched between the DLC and the metal substrate) to stop corrosion when immersed in a corrosive environment, by providing good adhesion and high load carrying capacity, and methods of making those systems. In a specific embodiment, a doped DLC top layer using different compounds such as Si or Ti are utilized to further enhance these properties. In one embodiment, the interface layer located between the substrate and the DLC layer includes SiₓN_{y} or TiN to improve adhesion of the DLC layer to the metal biomedical-grade substrate such as SS316L or Ti-alloy.

In one aspect, the present invention is directed to a medical device including a metal substrate, a DLC film and an intermediate layer located between the metal substrate and the DLC film. The intermediate layer includes a silicon material or a transition metal (such as Ti) material in a form such as a nitride, carbide or oxide.

The metal substrate is typically a biocompatible material. In one embodiment, the metal substrate includes stainless steel. In another embodiment, the metal substrate surface includes titanium or titanium alloy. In one embodiment, the intermediate layer includes silicon nitride, silicon carbide or silicon-carbon-nitride. In another embodiment the intermediate layer includes an organo-silicone. In another embodiment the intermediate layer includes transition metal nitride, carbide or oxide, specifically titanium nitride.

In one embodiment the DLC film includes a dopant, such as silicon, an organo-silicone or titanium. In one embodiment the dopant is present in the DLC film in an amount between about 2% to about 20% in an atomic amount.

In one embodiment the DLC film and the adhesion layer each have a thickness between about 2 nm and 10 micrometers, more specifically between about 50 nm and 1000 nm. Specifically, the DLC layer has a thickness between about 500nm and about 700 nm, and the adhesion layer has a thickness between about 300 nm and about 500 nm. In one embodiment, the intermediate layer is a multilayer structure. In another embodiment, the intermediate layer includes a gradient of at least two materials.

In another aspect, the present invention is directed to a medical device including a stainless steel substrate, a DLC layer comprising silicon in an amount between about 2 % and about 50% in an atomic amount, more specifically between about 2 % and about 20% in an atomic amount; and an intermediate layer including silicon nitride (SiₓN_{y}, such as Si₃N₄), silicon carbide (SiₓC_{y}), silicon carbonnitride (SiₓC_{y}N_{z}) or organosilicone, located between the stainless steel substrate and the DLC layer.

In another aspect, the present invention is directed to a method for making a medical device. The method includes providing a metal containing substrate, depositing an intermediate layer on the metal containing substrate and depositing a DLC layer on the intermediate layer. The intermediate layer includes a silicon containing material or a transition metal material, specifically a titanium containing material.

In one embodiment the DLC layer includes Si or Ti in an amount between about 2% and about 50% in an atomic amount, more specifically between about 2% and about 20% in an atomic amount.

In another embodiment, the intermediate layer includes silicon nitride, silicon carbide, silicon carbon nitride, organosilicone, or a transition metal material such as titanium nitride.

In another embodiment the metal containing substrate is stainless steel or a titanium alloy.

In another embodiment the intermediate layer and the DLC layer are deposited using plasma enhanced chemical vapor deposition.

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1 is a schematic cross-sectional view of an embodiment of a coating system with an interface configuration of the present invention.

FIG. 2 is a cross sectional SEM image of the SS\SiₓN_{y}\DLC system of Example 1 after coating.

FIG. 3 is an ERD compositional depth profile across the SS/SiₓN_{y}/DLC-Si sample of Example 2.

FIG. 4 is a graph showing the open circuit potential of the bare SS and SS\SiₓN_{y}\DLC samples during and after the sliding wear test of Example 1.

FIG. 5 is an SEM image of the wear track of the DLC system of Example 1 after the tribocorrosion experiment.

FIG. 6a is a graph showing the Bode plots obtained for the bare stainless steel substrate before and after the sliding wear test as described in Example 1. FIG. 6b is a graph showing the Bode plots obtained for the SS\SiₓN_{y}\DLC system before and after the sliding wear test as described in Example 1

FIG. 7 is a diagram showing the equivalent electrical circuit used to simulate the impedance spectra in Example 1.

FIG. 8 is an SEM image of a system having a nitrided interface layer after a corrosion wear test.

FIG. 9 is a cross-sectional SEM image of the SS316L/SiₓN_{y}/DLC(Si, 3.6 sccm of SiH₄) system described in Example 2 after coating before the corrosion test.

FIG. 10 is a graph showing the OCP of the SS316L/SiₓN_{y}/DLC(Si)(3.6 sccm of SiH₄) system before, during and after the wear test as described in Example 2.

FIG. 11 is a comparison of open circuit potential measurements of SS/DLC and SS/SiₓN_{y}/DLC-Si samples.

FIG. 12 is an SEM image of the SS316L/SiₓN_{y}/DLC(Si, 3.6 sccm of SiH₄) system described in Example 2 after the corrosion test.

FIG. 13 is a graph showing pitting potential of SS316L/SiₓN_{y}/DLC(Si), SS316L-nitrided/DLC and SS316L as described in Example 2.

FIG. 14 is a Bode diagram for bare stainless steel, DLC coated stainless steel with nitriding, and for DLC/Si with a silicon nitride intermediate layer as described in Example 2.

### DETAILED DESCRIPTION OF THE INVENTION

Amorphous diamond like carbon (DLC) material possesses unique physical, chemical, electrical, mechanical, tribological, optical and bio-medical characteristics. The present invention relates to the method of fabrication and to the interface architecture making the DLC coatings compatible with metal and metal-compound substrates, while simultaneously providing advantageous multifunctional surface characteristics including high wear, abrasion and scratch resistance, high corrosion resistance to aggressive environments, high surface resistivity, bio-compatibility and other technologically important properties.

As used herein, “diamond like carbon” or “DLC” means one or more of seven forms of amorphous carbon materials that display some of the unique properties of natural diamond. DLCs are metastable amorphous carbon materials containing both sp² and sp bonds. Depending on the ratio of sp² and sp³ bonds, DLCs can have a wide range of properties, including high hardness.

As used herein “tribo-corrosion” is intended to mean the chemical-electrochemical-mechanical process leading to a degradation of materials in sliding or rolling contacts immersed in a corrosive environment.

As used herein “plasma” refers to an ionized gas. “Ionized” means that at least one electron has been removed from a significant fraction of the molecules. The ionized gas contains free ions and electrons, and therefore is electrically conductive. Plasma also contains other excited species, such as free radicals and photons.

As used herein “substrate” refers to any surface.

The invention is described in further detail below with reference to the figures, in which like items are numbered the same in the several figures.

The implantable medical devices of the present invention include, but are not limited to, neurostimulators, catheters, cardiac valves, shunts, pacemakers, implantable cardioverter defibrillators, stimulation lead tips, medical electrodes, RF ablation devices, stents, stent grafts, drug-delivery devices, catheter tips, bone screws, bone covers, spinal plates, spinal rods, medical prosthesis, feeding tubes, trocar needles, clamps, forceps, guidewires, tissue cutting tools, vein harvesting tools, needles, lead anchors, fixation devices and the like. The methods of the present invention can be used in dental applications, such as but not limited to dental screws, dental wires, dental implants, artificial temporomandibular joint replacements, and the like. Further medical devices that can be effectively coated using the methods of the present invention including, but are not limited to, ophthalmic drug delivery devices, micro-abrasion devices, orthopedic implants, dental implants, dental wires, and surgical tools that require rough surfaces or non-slip surfaces. Ophthalmic drug delivery devices include, but are not limited to, ophthalmic rods. Ophthalmic rods are drug delivery devices for ophthalmic medications, and are typically long rods coated with an ocular diagnostic or therapeutic agent. The micro-abrasion devices are used in dental applications such as, but not limited to, tooth surfacing or surgical procedures such as, but not limited to, tissue roughening. Orthopedic implants include, but are not limited to, bone screws, plates, hip prosthesis, bone nails, wires, pins, artificial knee implants, tubular pins, and spinal implants

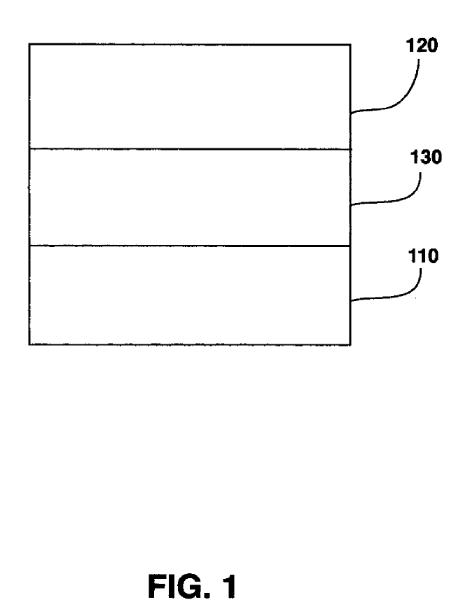

In one aspect, the invention is a new coating architecture as shown in FIG. 1, obtained by controlling the interfaces between a metal substrate 110 (such as stainless steel—SS, Ti-alloys, and others) and DLC coatings 120 in order to make it suitable for biomedical applications. In one embodiment, the invention includes a DLC layer 120, with an intermediate (such as a bonding, adhesion or sealing) layer 130 formed by Si or Ti based materials such as SiₓN_{y} or TiN coated onto the metal surface 110. In one embodiment, the DLC layer 120 is doped with a Si or Ti based material.

In one embodiment, the substrate is an implantable medical device having a biocompatible metal such as stainless steel, specifically SS-316, or titanium, specifically Ti-alloy, specifically Ti-6Al-4V.

The intermediate layer includes a silicon or transition metal material. Exemplary silicon materials include silicon nitride, organosilicone, silicon carbide and silicon carbon nitride. Exemplary transition metal materials include transition metal nitrides, carbides and oxides, such as titanium nitride. In one embodiment, the intermediate layer is silicon nitride. In one embodiment, the intermediate layer has a thickness between about 50 nm and about 1000 nm, more specifically between about 100 nm and about 800 nm, more specifically, between about 200 nm and about 600 nm, and more specifically between about 300 nm and about 500 nm. In another embodiment, the intermediate layer has a thickness greater than about 100 nm, more specifically, greater than about 200 nm, more specifically, greater than about 250 nm, more specifically, greater than about 300 nm, more specifically, greater than about 350 nm, more specifically, greater than about 400 nm, and more specifically, greater than about 450 nm. In another embodiment, the intermediate layer has a thickness less than about 1000 nm, more specifically, less than about 900 nm, more specifically, less than about 800 nm, more specifically, less than about 700 nm, more specifically, less than about 600 nm, more specifically, less than about 500 nm, and more specifically, less than about 400 nm.

In one embodiment, the intermediate layer can also be a gradient layer or a multilayer system including the materials described above, but featuring their sequence according to their microstructure, composition and/or their tribo-mechanical properties.

The use of an intermediate layer as described above increases adhesion of the DLC layer to the metal substrate compared to a DLC layer deposited directly to the metal substrate as demonstrated by microscratch test measurements.

In one embodiment, the DLC layer has a thickness between about 50 nm and about 1000 nm, more specifically between about 100 nm and about 800 nm, more specifically between about 300 nm and about 800 nm, more specifically between about 400 nm and about 800 nm, and more specifically between about 500 nm and about 700 nm. In another embodiment, the DLC layer has a thickness greater than about 100 nm, more specifically, greater than about 200 nm, more specifically, greater than about 300 nm, more specifically, greater than about 400 nm, more specifically, greater than about 500 nm, and more specifically, greater than about 600 nm. In another embodiment, the DLC layer has a thickness less than about 1000 nm, more specifically, less than about 900 nm, more specifically, less than about 800 nm, and more specifically, less than about 700 nm.

The DLC layer can optionally include a dopant, such as silicon, organosilicone or a transition metal, such as titanium. The dopant is present in the DLC film in an amount between about 1% to about 50% in an atomic amount, more specifically in an amount between about 2% to about 30% in an atomic amount, and more specifically, in an amount between about 2% to about 20% in an atomic amount, and more specifically, in an amount between about 2% and about 10% in an atomic amount.

The use of Si-doped DLC (DLC-Si) can provide further improvement of adhesion for DLC coatings to metal substrates, reduce the film's mechanical stress and/or increase density.

A specific embodiment includes a stainless steel/Si₃N₄/DLC-Si system with a Si concentration in the DLC layer between 2% and 20% in an atomic amount.

In one embodiment, the coated devices of the present invention, such as implantable medical devices, are made by providing a metal containing substrate, depositing an intermediate layer on the metal containing substrate, wherein the intermediate layer comprises a silicon containing material or a titanium containing material, and depositing a DLC layer on the intermediate layer.

The metal containing substrate, such as stainless steel, is preferably cleaned prior to deposition. This can be carried out by many different ways known to those of skill in the art. In an exemplary embodiment, the substrate is mechanically polished using an appropriate polishing media, such as a 1 μm alumina suspension. After polishing, the substrate can be ultrasonically cleaned in an appropriate solvent, such as acetone and isopropanol. The substrate can be additionally cleaned in a deposition chamber, for example with Ar plasma sputtering to remove any native oxide formed after polishing.

After cleaning, the intermediate layer is deposited on the substrate using coating techniques known to those of skill in the art. In an exemplary embodiment, the intermediate layer is deposited using plasma enhanced chemical vapor deposition (PECVD) under appropriate coating conditions which include temperature in the range of room temperature (20° C.) to about 900° C., more specifically in the range of room temperature to about 450° C., and even more specifically in the range of room temperature to about 300° C., process pressure in the range of 10⁻⁴ to about 10⁻¹ Torr, time in the range of about 5 seconds to about 5 hours, and bias voltage in the range of 0 to about −1,200 V.

After the deposition of the intermediate layer, the DLC layer is deposited on the intermediate layer using coating techniques known to those of skill in the art. In an exemplary embodiment, the DLC layer is deposited using PECVD under appropriate coating conditions which include temperature in the range of room temperature (˜20° C.) to about 900° C., more specifically in the range of room temperature to about 450° C., and even more specifically in the range of room temperature to about 300° C., process pressure in the range of 10⁻⁴ to about 10⁻¹ Torr, time in the range of about 5 seconds to about 5 hours, and bias voltage in the range of 0 to about −1,200 V.

As discussed above, in one embodiment, film deposition is performed by a radiofrequency PECVD process. In one embodiment, a turbomolecularly pumped vacuum chamber (about 40 cm in diameter and 30 cm high) is equipped with a radio frequency-powered (RF, 13.56 MHz) substrate holder (15 cm in diameter) on which a negative substrate bias, VB, develops. This allows for the optimization of deposition conditions of individual films and hence their microstructure by controlling the energy and flux of impinging ions in conjunction with the kind of materials specific for each system.

The following examples illustrate the principles and advantages of the invention.

### EXAMPLES

### Example 1

### SS/SiₓN_{y}/DLC

### Coating Depositions

DLC coatings were deposited onto SS316L substrates (25×25×1.2 mm) using a turbomolecularly pumped radio frequency (RF, 13.56 MHz) PECVD system, equipped with a 15 cm diameter RF powered electrode where a self-induced DC bias voltage, VB, developed. An amorphous hydrogenated silicon nitride (a-SiN_{y}:H) bond layer was deposited as an intermediate layer between the SS316L substrate and the DLC film.

A SS316L substrate was mechanically polished using a 1 μm alumina suspension. After polishing, the specimen was ultrasonically cleaned in acetone (15 minutes) and isopropanol (15 minutes), and then introduced into the deposition chamber. Prior to deposition, the substrate was cleaned with Ar plasma sputtering for 15 minutes to remove any native oxide formed after polishing.

The SiₓN_{y}, or more specifically a-SiN_{y}:H bond layer was deposited using a gas mixture of silane (SiH₄), nitrogen (N₂) and Ar at a total working pressure of 100 mTorr and a bias voltage of −400V for 10 minutes. The thickness of this layer was approximately 350 nm at a substrate temperature close to the room temperature or slightly higher, but not exceeding 200° C.

The DLC film was then deposited using a gas mixture of methane (CH₄) and Argon (Ar) at a total working pressure of 100 mTorr and a bias voltage of −500V for 30 minutes. The thickness of the DLC was approximately 650 nm at a temperature close to the room temperature or slightly higher, but not exceeding 200° C.

The conditions of the substrate pretreatment, the intermediate layer deposition and the DLC film deposition are shown in Table 1, as well as the mechanical properties measured using a nano-indentation (depth-sensing indentation) technique using a Tribonindenter instrument manufactured by Hysitron Inc., using the Oliver and Pharr approach as described in W. C. Oliver, G. M. Pharr, J. Mater. Res. 7 (1992) 1564. The chemical composition of the DLC and the a-SiN_{y}:H film, measured using an Elastic Recoil Detection (ERD) technique as described in S. C. Gujrathi, E. Sacher, J. J. Pireaux and S. P. Kowalczyk, Editors, Metallized Polymers, Vol. 440, ACS Symposium Series, ACS Washington D.C. (1990) p.88., are shown in Table 2.

TABLE 1

| | | | Bias | | | | Young's |
|---|---|---|---|---|---|---|---|
| | Pressure | Gas mixture | voltage | Duration | Thickness | Hardness | Modulus |
| Process | mTorr | sccm | V_{B} | min | nm | GPa | GPa |
| Sputter- | 100 | Ar (10) | −800 | 15 | — | 6 | 220 |
| cleaning | | | | | | | |
| a:SiN_{y}-H | 100 | SiH₄ (6.6), | −400 | 10 | 350 | 17 | 160 |
| deposition | | N₂ (20), | | | | | |
| | | Ar (30) | | | | | |
| DLC | 100 | CH₄ (40), | −500 | 30 | 650 | 18 | 140 |
| deposition | | Ar (10) | | | | | |

TABLE 2

| | C (at. %) | Si (at. %) | N (at. %) | H (at. %) |
|---|---|---|---|---|
| DLC | 85 | — | — | 15 |
| a-SiN:H | — | 35 | 48 | 17 |

### Surface Characterization

A Field Emission Scanning Electron Microscope (FESEM) Philips XL30 equipped with an Energy Dispersive Spectrometer (EDS) was used to characterize the surface morphology and to perform the elemental analysis of the worn surfaces. SEM was also used to perform the cross-section analysis of the as-deposited surfaces.

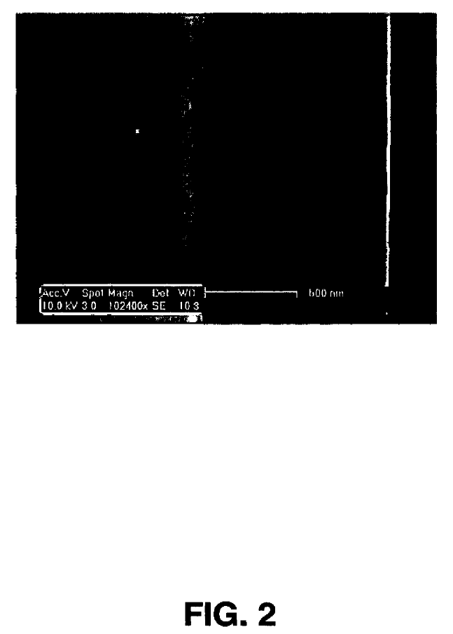

FIG. 2 shows the cross section images of the SSSiₓN_{y}\DLC specimen. The thickness of the SixNylayer 200 was approximately 350 nm. The thickness of the DLC layer 210 was approximately 650 nm.

### Tribocorrosion Test

Tribo-corrosion properties were assessed by the tribo-electrochemical techniques using an electrochemical set-up (mainly of the three-electrode type) for controlling the potential of the surface of a conducting material subjected to rubbing in a tribometer. In this way it is possible to carry out friction and wear tests in an electrolytic solution under well-defined electrochemical conditions determined by the applied electrode potential. In addition, these techniques offer the possibility to evaluate in-situ and in real time the kinetics of electrochemical oxidation reactions (corrosion) by the measurement of an electric current.

Tribocorrosion experiments were performed using a linear reciprocating ball-on-plate tribometer. Here, a 3/16″ (4.75 mm) diameter alumina ball was rubbed on the specimen surface immersed in the test solution. The sample served as the working electrode and its potential was controlled using an Autolab PGSTAT302 potentiostat equipped with a Frequency Response Analyzer. The counter electrode was made of coiled platinum and the standard calomel electrode SCE (+241 mV versus standard hydrogen electrode) was used as a reference electrode. The normal force was applied using a compression spring.

Sliding wear experiments were carried out with 9 N normal force, which corresponds to a maximum Hertzian contact pressure of 1.18 GPa, calculated using the above mentioned alumina ball and a SS316L flat surface. The stroke length was 10 mm and the sliding frequency was 1 Hz. Ringer's solution of pH 6.6 was used as an electrolyte: its composition was 9 g/l NaCl, 0.4 g/l KCl, 0.17 g/l CaCI₂ and 2.1 g/l NaHCO₃ in water.

Electrochemical set-up with three electrodes was used for controlling and measuring the potential of a surface subjected to classical sliding wear testing. Open circuit potential (OCP) measurements during sliding wear were used to follow in situ the tribocorrosion behavior of the DLC-coated stainless steel surfaces. Electrochemical Impedance Spectroscopy (EIS) was used to characterize the electrochemical behavior of the surfaces before and after sliding wear. OCP measurements were shown to reflect the coating behavior during the sliding wear test, a decrease in the OCP during sliding wear indicated the removal of the coating and the exposure of the substrate to the electrolyte. The results showed that the DLC coating resisted the entire tribocorrosion test (1800 cycles) without failure when the a-SiNy:H bond layer was used. The EIS results are interpreted in terms of appropriate equivalent circuits. It is believed that the a-SiN_{y}:H bond layer significantly decreased the charge transfer between the substrate and the electrolyte by acting as a corrosion barrier; the resistance to charge transfer of DLC-coated stainless steel with a-SiNY:H bond layer was found to be approximately 2 GΩ/cm².

The sequence of operations during the tribocorrosion test is as follows: First, the sample was immersed in the Ringer's solution for 1 hour in order to reach a stable potential. Next, EIS was performed to characterize the electrochemical behavior of the surface before sliding wear. Then, the alumina ball was loaded on the sample surface and the sliding wear test was started. The sliding was stopped after 1800 cycles. During and after the sliding wear test, the OCP was continuously monitored. Finally, EIS was performed to characterize the electrochemical behavior of the surface after sliding wear. The EIS spectra were acquired over the frequency range 10⁵ Hz-10⁻² Hz, at the OCP, with an AC amplitude of 10 mV.

The tribocorrosion experiments were repeated three times for the same surface condition to validate the results. SS and SS\SiₓN_{y}\DLC notations are used to represent the above mentioned samples.

Open Circuit Potential (OCP) Measurement during the Tribocorrosion Test

### Bare Stainless Steel

During the first hour of immersion in the electrolyte, the open circuit potential OCP increased and stabilized at approximately −90 mV. It is believed that the naturally occurred oxide layer protecting the stainless steel 316L is very stable and offers an excellent corrosion resistance to the stainless steel substrate in aerated solution.

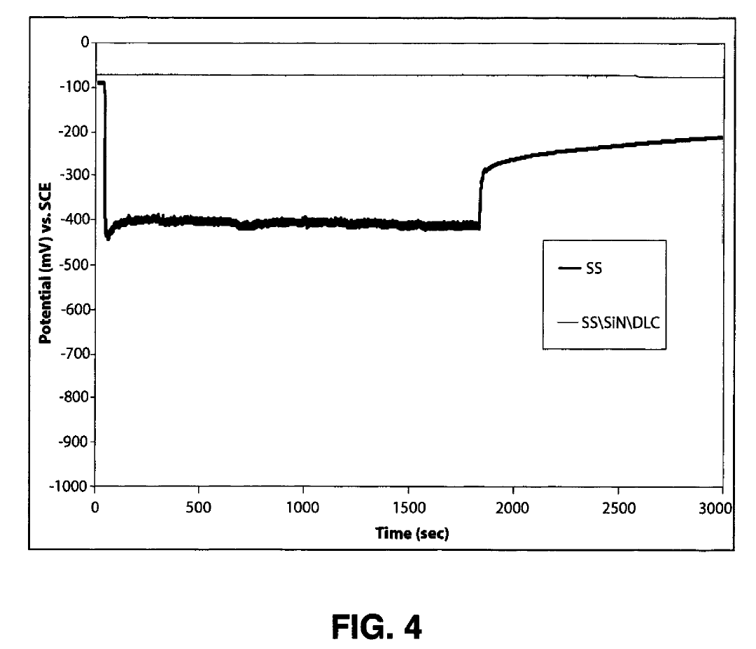

At the time the sliding wear experiment started, a sudden decrease of the OCP took place, the potential shifted negatively from −90 mV to approximately −400 mV as shown by curve 400 in FIG. 4. During the sliding cycles, the potential was fluctuating in phase with the reciprocating motion of the alumina ball. When the sliding wear cycles were stopped, the OCP first increased steeply and then returned progressively to a steady state value. During the sliding wear test the friction coefficient was approximately 0.3.

It is believed that the increase of the OCP after the sliding wear cycles is due to the repassivation of the stainless steel surface. It is believed that in the absence of sliding wear, the stainless steel surface repassivates and the OCP increases. DLC-coated stainless steel with a-SiN_{y}:H bond layer (SS\SiₓN_{y}\DLC)

The OCP during the first hour immersion of the SS\SiₓN_{y}\DLC stabilized at approximately −70 mV. The potential remained constant during and after the sliding wear test as shown by curve 410 in FIG. 4 and the fluctuations of the potential during the sliding wear were small compared to the fluctuations on the other samples. In addition, the friction coefficient was constant, 0.08, throughout the entire sliding wear test. This indicates that the active, metallic stainless steel material was not exposed to the aqueous environment during the sliding wear test and that the alumina ball was rubbing against the DLC coating.

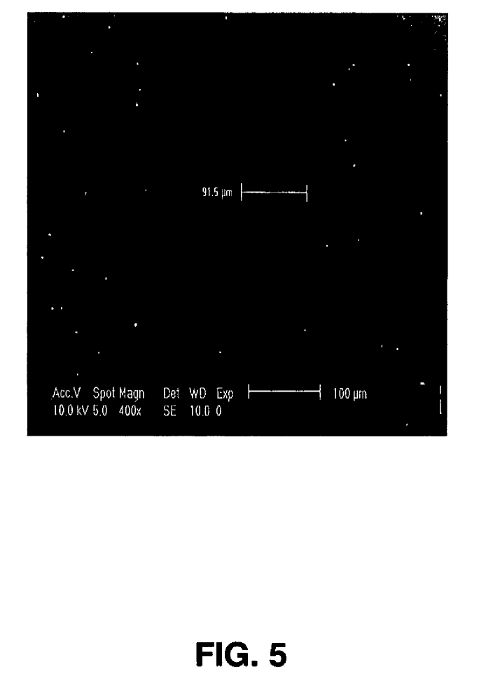

FIG. 5 shows the SEM image of the wear track after the tribocorrosion experiment. It can be seen that the DLC layer was not removed from the wear track. The EDS spectrum acquired from the wear track also confirmed the presence of the DLC layer after the tribocorrosion experiment.

It is believed that the SixNy layer significantly reduced the charge transfer between the metallic substrate and the electrolyte, by acting as a barrier layer. Therefore it reduced the possibility of weakening of the interface between the substrate and the film. The SixNylayer seems to significantly reduce the corrosion processes, and the DLC layer, due to its high hardness and wear resistance, appears to protect the SiₓN_{y} and the stainless steel substrate from mechanical wear.

### Electrochemical Impedance Spectroscopv

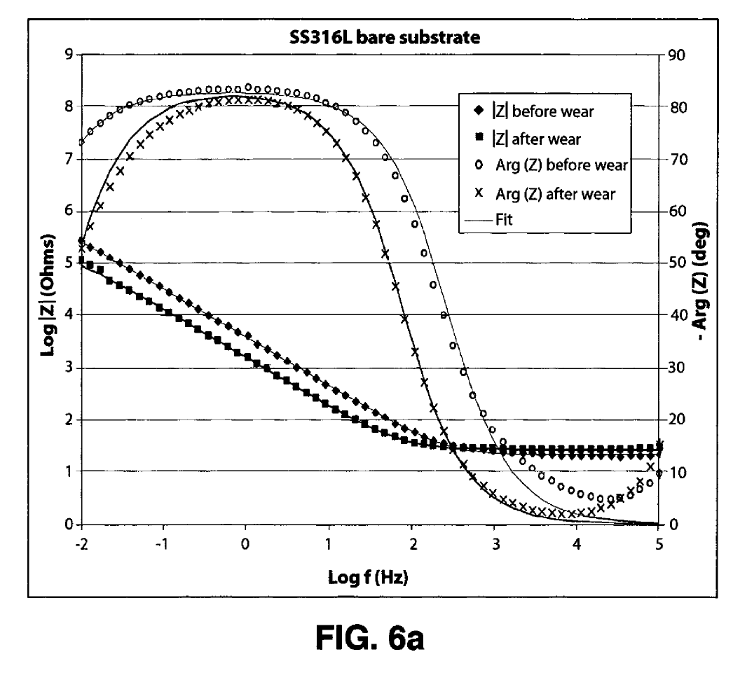

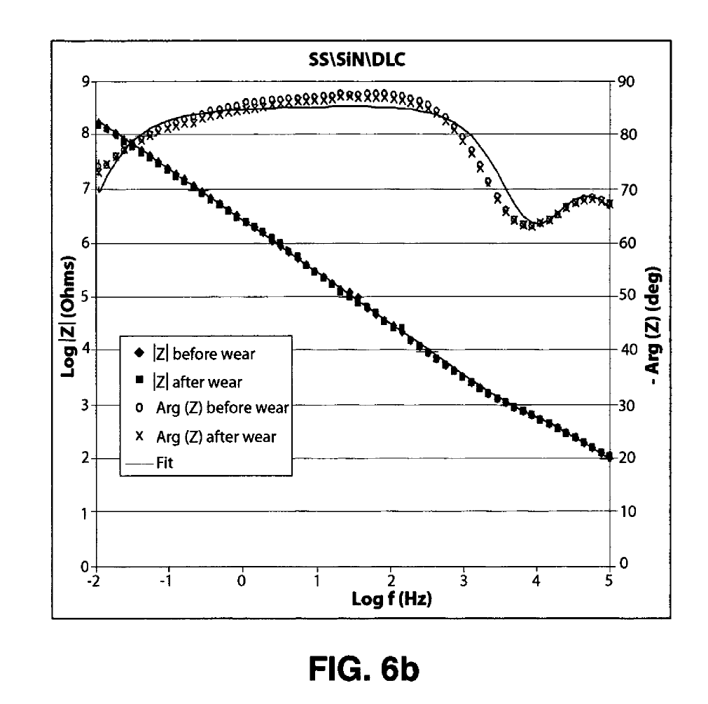

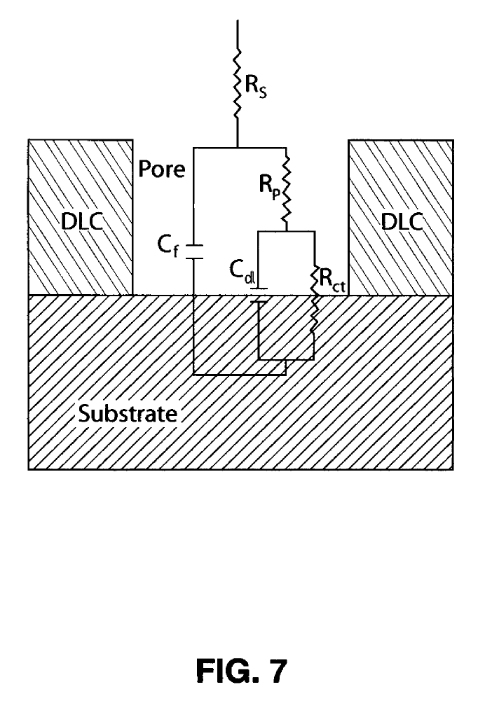

Electrochemical impedance spectroscopy EIS was performed before and after the sliding wear test. The samples were not removed from the solution between the two measurements so that the sample surfaces would not be affected by oxygen. The EIS results are presented using Bode plots as shown in FIGS. 6a and 6b, and the obtained spectra were interpreted in terms of appropriate equivalent circuits, shown in FIG. 7, acquired before and after sliding wear, of the SS and SS\SiₓN_{y}\DLC as well as the fitting curves obtained from the equivalent electrical circuits. The data obtained from the EIS spectra simulation are shown below in Table 3. The results are not normalized for the working electrode area (2.83 cm²) because the surface was not homogeneous after the sliding wear test; it consisted of worn and unworn areas

### Bare Stainless Steel

The Bode plots obtained for the bare stainless steel substrate before and after the sliding wear test are shown in FIG. 6a. The plots indicate one time constant as one peak was observed in the Arg (Z) vs. f plot. Using the Randle circuit formalism for simulation, the resistance to charge transfer Rt was 838 kΩ before the sliding wear, it became 177 kΩ after, as shown in Table 3. Before sliding wear the surface was homogeneous and therefore the R_{ct} could be normalized for the electrode area to give 2,371 kOhm/cm². After sliding wear, the surface is considered to consist of a parallel connection of two different areas with two different impedances: the wear track, where a new passive layer was formed after the sliding wear test with impedance Z_{w}, and the unworn area where the passive film was not destroyed with impedance Z_{uw}. The global impedance is then

1/Z=1/Z_{w}+1/Z_{uw}

It has been demonstrated that a similar relation can be derived for the resistance to charge transfer R_{ct}, R_{ctw}, and R_{ctuw} (charge transfer relative to the total area, the wear track area and unworn area, respectively).

1/R_{ct}=1/R_{ctw}+1/R_{ctuw}

Where R_{ct}=177 KΩ.

Knowing from the SEM image that the wear track area is approximately 0.03 cm² and consequently the unworn area is 2.8 cm², and assuming that the electrochemical activities of the unworn area before and after sliding wear are the same (R_{ctuw}=2,371/2.8=847 kΩ), R_{ctw} can be calculated; it was found to be 224 kΩ or 6.72 kΩ/cm² after normalization, compared to 2,363 kΩ/cm² for the unworn surface. This difference indicated that the passive film formed in the active area of the wear track after sliding wear had not regained its initial thickness and protective properties. Indeed, after stabilization of the worn sample for 24 hours in the solution, EIS was performed and the results showed that Rt of the total area was very close to Rt calculated before sliding wear.

DLC-Coated SS316L with a:SiN_{y}-H Bond Layer (SS\SiₓN_{y}\DLC)

The Bode plots obtained for the SS\SiₓN_{y}\DLC before and after the sliding wear test are shown in FIG. 6b. As it can be seen, the impedance increased significantly with the insertion of the a-SiN_{y}:H bond layer and the log(Z) plot shifted upward at high frequencies indicating more capacitive response of the coating. Also, the two spectra acquired before and after sliding wear were similar indicating that the electrochemical behavior of the surface was not affected by the sliding wear. In other terms, the SiₓN_{y}\DLC layer was protecting the stainless steel surface after sliding wear in the same manner as it was before. The electrical circuit used to simulate the impedance spectra is shown in FIG. 7. It includes the following elements; Rₛ 702 corresponds to the solution resistance, C_{f} 704 is the capacitance of the DLC film 712, Rₚ 706 is the solution resistance inside the pores, R_{ct} 708 and C_{d1} 710 are resistance to charge transfer and the double layer capacitance at the contact between the electrolyte and the substrate 714 inside the pores. It should be mentioned that C_{f} 704 and C_{d1} 710 are replaced by the constant phase element (non-ideal capacitor) assigned Q_{f} and Q_{d1}.

Using this circuit, it was found, as shown in Table 3, that R_{ct} was equal to 2 GOhm/cm² approximately before and after sliding wear compared to 30.4 MOhm/cm² for the DLC without a-SiN_{y}:H layer indicating a significant improvement in the corrosion properties of the film. In addition, the solution resistance in the pores, Rp, was found to be 1.4 kΩ (3.96 kΩ/cm²). This high value reflected the high resistance against the infiltration of the liquid solution through the film to reach the substrate.

These results showed that a-SiN_{y}:H layer provides an excellent barrier against charge transfer by significantly reducing the contact between the solution and the substrate.

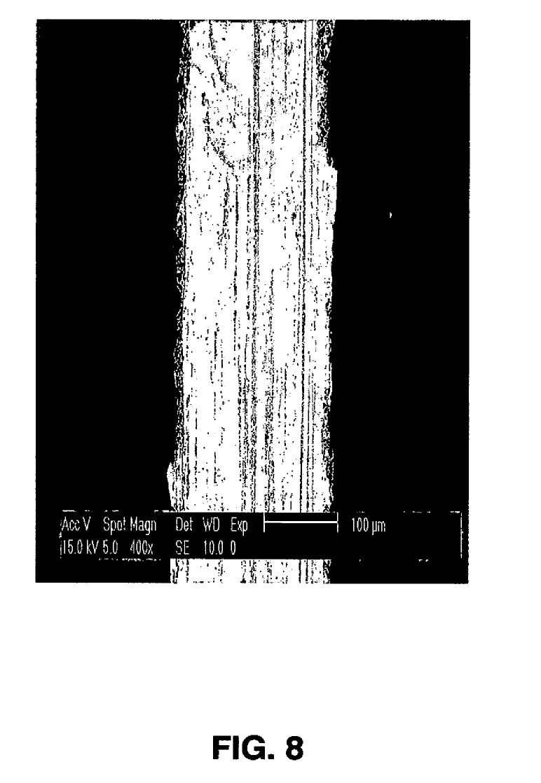

A nitrided interface layer alone between the SS316L substrate and DLC did not provide enough adhesion to withstand the corrosive wear test. The white track in FIG. 8 shows that the DLC is entirely removed. Moreover, the EDS spectra acquired from the wear track confirmed the removal of the DLC.

TABLE 3
The data obtained from the EIS spectra simulation (exposed
area 2.83 cm²).

| | Rₛ | R_{ct} | Rₚ |
|---|---|---|---|
| Sample | (Ω) | (Ω) | (Ω) |
| SS | 12 | 838 × 10³ | — |
| Before wear | | | |
| SS | 13 | 177 × 10³ | — |
| After wear | | | |
| SS\SiₓN_{y}\DLC | 22 | 708 × 10⁶ | 1400 |
| before & | | | |
| after wear | | | |

### Example 2

A SS316L substrate was prepared and the a-SiN_{y}:H intermediate layer was deposited as described in Example 1. The DLC layer was deposited under the conditions as described above with an additional flow rate of 3.6 sccm of SiH₄ resulting in a DLC layer doped with Si.

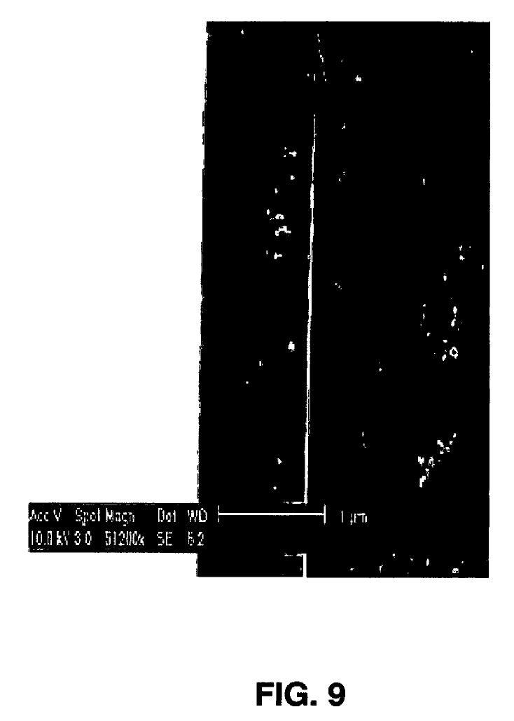

Cross section analysis by SEM as shown in FIG. 9 confirmed a total coating thickness of about 1 μm.

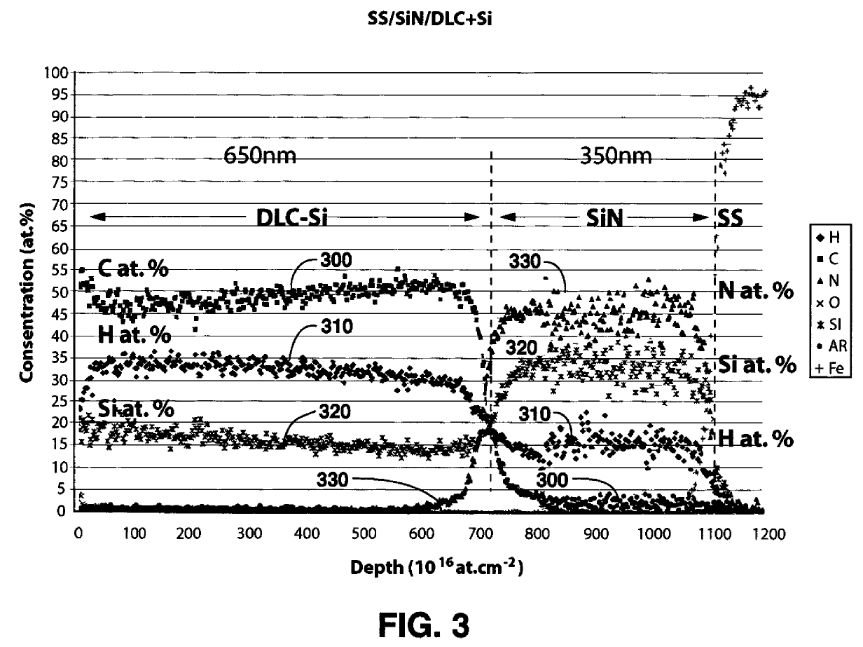

FIG. 3 shows an ERD compositional depth profile indicating the atomic concentration of the participating elements C 300, H 310, Si 320 and N 330. The profile of FIG. 3 shows the composition of the intermediate silicon nitride layer 340 (350 nm) and the subsequent DLC layer 350 (650 nm).

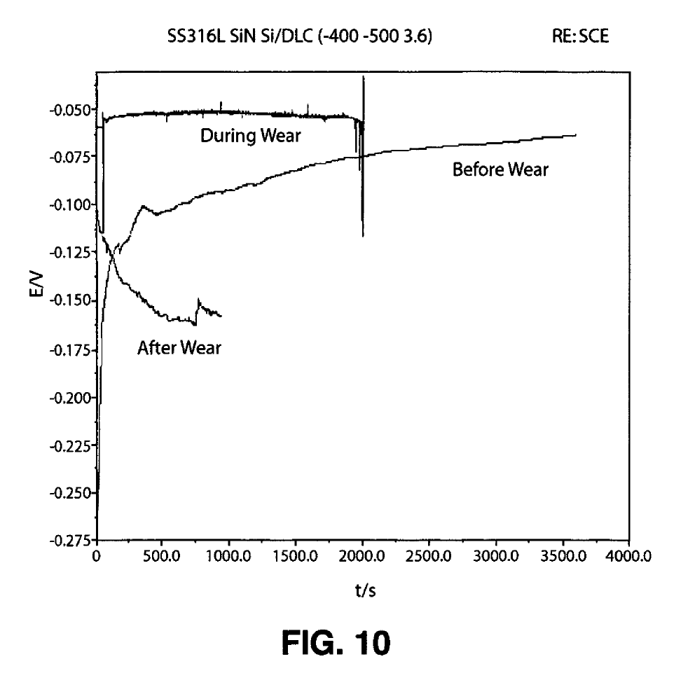

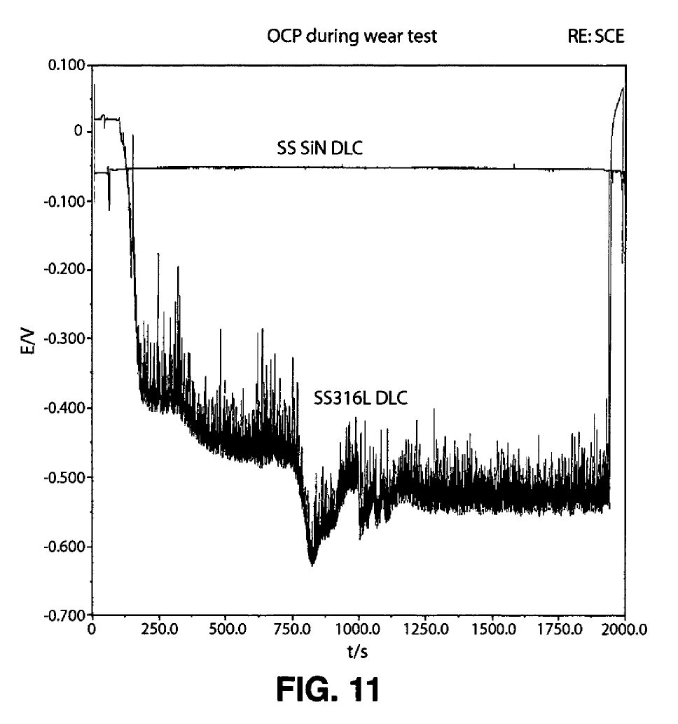

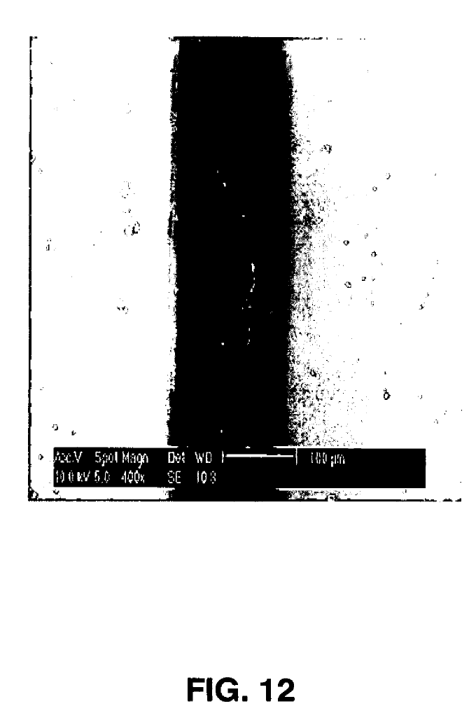

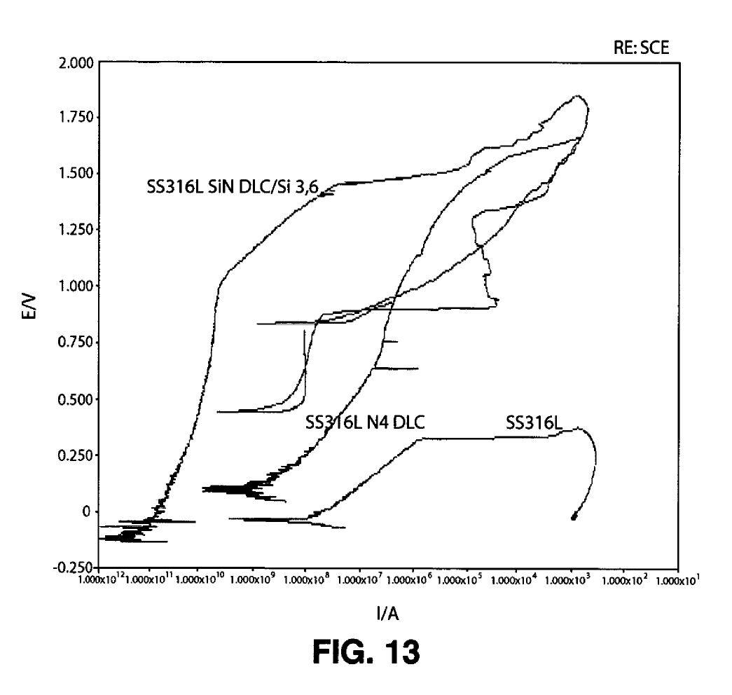

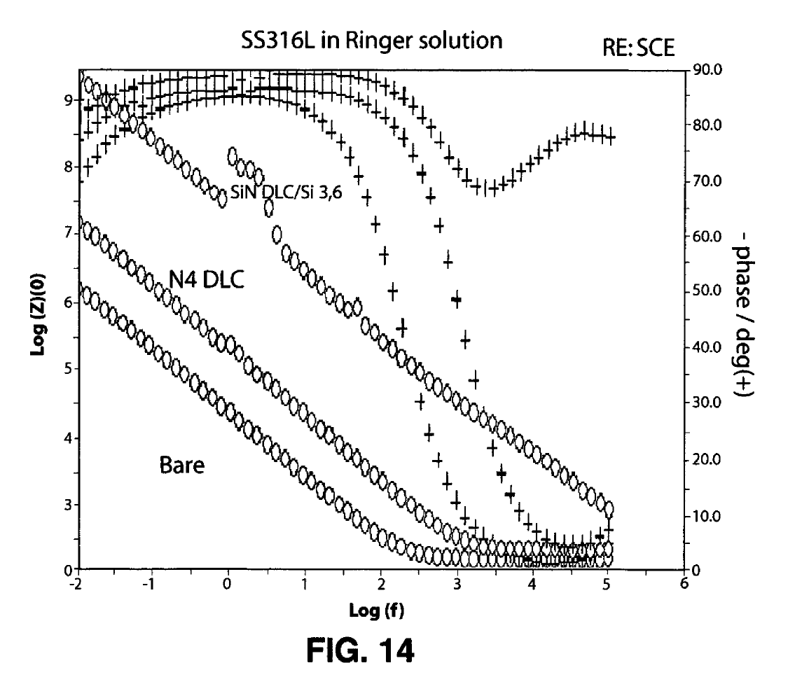

The sample was subjected to tribological testing as described above. The darker track in FIG. 12 shows that the DLC is still there on the wear track, FIG. 10 shows the OCP measurements before, during and after wear test, FIG. 11 shows a comparison of the DLC on SS with that including the intermediate layer, FIG. 12 shows an SEM image after tribo-wear, FIG. 13 shows the corrosion current as well as the passivation current, and FIG. 14 shows the EIS measurement before and after the wear test.

Adhesion having a critical load of 9.6N was obtained for this system: SS316L/SiₓN_{y} (bias=−400V, thickness=350 nm) / DLC(Si) (bias=−500V, thickness=650 nm, SiH₄ flow=3.6 sccm) using the microstcratch test.

DLC coatings with a silicon nitride intermediate layer significantly improved the corrosion resistance of the stainless steel and DLC coatings with a nitrided interface layer. The corrosion current as well as the passivation current were reduced by approximately three orders of magnitude, and the pitting potential for the coated sample is more than 1 V compared to 0.35V for the bare stainless steel as shown in FIG. 13.

EIS measurements were also performed for the bare stainless steel 1410, the DLC coated stainless steel after 4 hours of nitriding 1420, and the DLC(Si) coated (3.6 sccm SiH₄) stainless steel with a SixNy intermediate layer 1430. FIG. 14 shows the results in Bode plot mode. The circles correspond to the module of the impedance (in log scale) and the crosses correspond to the phase difference of the impedance. The addition of DLC without SiₓN_{y} increased the impedance by one order of magnitude. However, the addition of DLC/Si with a SiₓN_{y} intermediate layer increased the impedance by 3 orders of magnitude. In addition, the phase difference for the sample with silicon nitride was almost 90 degrees over the frequency range which indicated that the silicon nitride has a capacitive behavior and that its resistance to charge transfer extremely high.

In light of the detailed description of the invention and the examples presented above, it can be appreciated that the several objects of the invention are achieved.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

We claim:

1. A medical device comprising:
a metal substrate;
a diamond-like carbon (DLC) film; and
an intermediate layer located between the metal substrate and the DLC film, the intermediate layer comprising a silicon material or a titanium material.

2. The device of claim 1, wherein the metal substrate surface comprises stainless steel.

3. The device of claim 1, wherein the metal substrate surface comprises titanium.

4. The device of claim 1, wherein the intermediate layer comprises a material selected from the group consisting of an organosilicone, silicon nitride, silicon carbide, silicon carbon nitride, and transition metal nitrides.

5. The device of claim 4, wherein the intermediate layer comprises silicon nitride.

6. The device of claim 4, wherein the intermediate layer comprises titanium nitride.

7. The device of claim 1, wherein the DLC film comprises a dopant.

8. The device of claim 7, wherein the dopant comprises silicon.

9. The device of claim 7, wherein the dopant comprises an organo-silicone.

10. The device of claim 7, wherein the dopant comprises a transition metal.

11. The device of claim 7, wherein the dopant is present in the DLC film in an amount between about 2% to about 20% in an atomic amount.

12. The device of claim 1, wherein the DLC film and the adhesion layer each have a thickness between about 50 nm and 1000 nm.

13. The device of claim 12, wherein the DLC film has a thickness between about 500 nm and about 700 nm.

14. The device of claim 12, wherein the adhesion layer has a thickness between about 300 nm and about 500 nm.

15. The device of claim 1, wherein the intermediate layer is a multilayer structure.

16. The device of claim 1, wherein the intermediate layer comprises a gradient of at least two materials.

17. A medical device comprising:
a stainless steel substrate;
a DLC layer comprising silicon in an amount between about 2% to about 20% in an atomic amount; and
an intermediate layer comprising SiₓN_{y} located between the stainless steel substrate and the DLC layer.

18. A method for making a medical device, the method comprising
providing a metal containing substrate;
depositing an intermediate layer on the metal containing substrate, wherein the intermediate layer comprises a silicon containing material or a titanium containing material; and thereafter
depositing a DLC layer on the intermediate layer.

19. The method of claim 16, wherein the DLC layer comprises Si or Ti in an amount between about 2% and about 20% in an atomic amount.

20. The method of claim 16, wherein the intermediate layer comprises a material selected from the group consisting of silicon nitride, silicon carbide, silicon carbon nitride, organosilicone, and titanium nitride.
